(19) United States
(12) Patent Application Publication
(10) Pub. No.: US 2007/0239038 A1
(43) Pub. Date: Oct. 11, 2007

(54) Method and apparatus for monitoring heat stress

(76) Inventors: Ion V. Nicolaescu, Carpentersville, IL (US); Lilliana Grajales, Bloomingdale, IL (US)

Correspondence Address: MOTOROLA, INC., 1303 EAST ALGONQUIN ROAD, IL01/3RD, SCHAUMBURG, IL, 60196, US

(21) Appl. No.: 11/390,719

(22) Filed: Mar. 28, 2006

(51) Int. Cl. A61B 5/02; A61B 5/00

(52) U.S. Cl. 600/483; 600/549

(57) ABSTRACT

A method and apparatus for monitoring heat stress, wherein the apparatus may include a skin surface temperature probe and heart rate probe that do not require direct physical contact to a user's skin. The method includes the steps of: measuring a plurality of physiological parameters including at least heart rate and skin surface temperature of a user; calculating at least body core temperature of the user as a function of the plurality of physiological parameters using a predetermined algorithm; comparing at least heart rate and body core temperature to corresponding maximum thresholds; and alerting the user when at least one of heart rate and body core temperature exceeds the corresponding maximum threshold.

### FIELD OF THE INVENTION

The present invention relates generally to monitoring physiological parameters, and more specifically to measuring and monitoring heat stress for a user of heat stress monitoring apparatus and alerting the user of when to exit an environment to minimize the user's risk for developing heat-related disorders.

### BACKGROUND OF THE INVENTION

Heat stress is the buildup of heat in a body that is generated by the muscles of the body at work, which can be intensified by environmental conditions such as, for instance, air temperature, the temperature of surrounding objects, humidity, and air movement. When the body is subjected to more heat than it can cope with, various heat-related disorders can result (e.g., dehydration, heat exhaustion, heat stroke, etc.), some of which can lead to death. Workers such as firefighters, steel workers and miners, for example, are frequently required to perform high effort activity in hot environments, putting them at risk of developing heat-related disorders. Moreover, aging people or people with certain health conditions can be at a high risk for developing heat-related disorders even without much physical exertion.

It is well known that a body core temperature above 40 degrees Celsius puts someone in danger of heat stroke. Body core temperature as used herein is the mean temperature of the tissues at a depth below that which is affected directly by a change in the temperature gradient through peripheral tissues. Deep tissues include, for example, tissues of internal organs such as the heart, lungs and other vital organs. Peripheral tissues include tissues of the periphery of the body including, for example, tissues of the arms, legs and close to the skin. To minimize the risk of developing heat-related disorders, the Occupational Health and Safety Administration (OHSA) recommends that body core temperature not be allowed to increase above 38 degrees Celsius for extended periods of time.

Thus for persons at risk of developing heat-related disorders, for instance based upon their occupation, it is desirable to have a method and apparatus for monitoring physiological parameters such as body core temperature and heart rate so that a user of the apparatus (and perhaps additional persons) may be alerted of when the user should exit an environment to decrease his or her risk for developing heat-related disorders. It is further desirable that the apparatus be adaptable to include at least some sensors that do not require direct physical contact to the user's skin for proper functioning and that an accurate measure of body core temperature be attainable without requiring the use of invasive sensors.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates a heat balance model used in determining parameters for monitoring heat stress in accordance with embodiments of the present invention;

FIG. 2 illustrates a method for monitoring heat stress in accordance with embodiments of the present invention;

FIG. 3 illustrates a block diagram of elements of a wearable physiological monitoring system embodying the present invention;

FIG. 4 illustrates a schematic diagram of a wearable harness embodying the present invention;

FIG. 5 illustrates a schematic diagram of a vest embodying the present invention;

FIG. 6 illustrates a temperature probe in accordance with embodiments of the present invention; and

FIG. 7 illustrates a circuit diagram describing the operation of the temperature probe of FIG. 6.

### DETAILED DESCRIPTION OF THE INVENTION

Before describing in detail embodiments that are in accordance with the present invention, it should be observed that the embodiments reside primarily in combinations of method steps and apparatus components related to a method and apparatus for monitoring heat stress. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Thus, it will be appreciated that for simplicity and clarity of illustration, common and well-understood elements that are useful or necessary in a commercially feasible embodiment may not be depicted in order to facilitate a less obstructed view of these various embodiments.

It will be appreciated that embodiments of the invention described herein may be comprised of one or more conventional processors and unique stored program instructions that control the one or more processors to implement, in conjunction with certain non-processor circuits, some, most, or all of the functions of the method and apparatus for heat stress monitoring described herein. The non-processor circuits may include, but are not limited to, a radio receiver, a radio transmitter and power source circuits. As such, these functions may be interpreted as steps of a method to perform the heat stress monitoring described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Thus, methods and means for these functions have been described herein. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

Generally speaking, pursuant to the various embodiments, a method and apparatus for monitoring heat stress is described. The method takes into consideration a plurality of measured physiological parameters for a user including the user's heart rate and skin surface temperature and further (optionally) factors in personal characteristics associated with the user such as height, weight, age, acclimatization, etc., to calculate the user's body core temperature. A physiological parameter as used herein is any parameter being in accord with or characteristic of the normal functioning of a living organism. The user's heart rate and body core temperature are monitored and compared to respective thresholds to determine when to alert the user so as to minimize the user's risk of developing one or more heat related disorders in a given environment.

The method is implemented in a system that includes: a plurality of sensors for measuring the physiological parameters being utilized in the method; a processor for performing the method; and alarm apparatus for alerting the user when a monitored physiological parameter exceeds a threshold. In one embodiment, at least one of the sensors does not require direct physical contact with the user's skin to measure the corresponding physiological parameter, which makes the apparatus suitable to many more applications than apparatus that requires skin contact, especially, apparatus that requires invasive sensors.

For example, in accordance with one embodiment of the invention is a skin surface temperature probe that includes: a first portion comprising a thermally conductive material having a first plurality of temperature measuring devices coupled thereto and having a first face adjacent to a first surface of the sensor; a second portion comprising a thermally conductive material having a second plurality of temperature measuring devices coupled thereto and having a second face adjacent to the first surface of the sensor; and a third portion coupled between the first and second portions and substantially thermally isolating the first portion from the second portion, wherein upon the first surface of the sensor contacting a second surface that is adjacent to a user's skin surface a difference in heat flux between the first and second portions is generated and measurable based on the first and second plurality of temperature measuring devices, the measurable difference in heat flux for use in determining skin surface temperature of the user. In one embodiment, the temperature sensing devices are thermistors.

Those skilled in the art will realize that the above recognized advantages and other advantages described herein are merely exemplary and are not meant to be a complete rendering of all of the advantages of the various embodiments of the present invention.

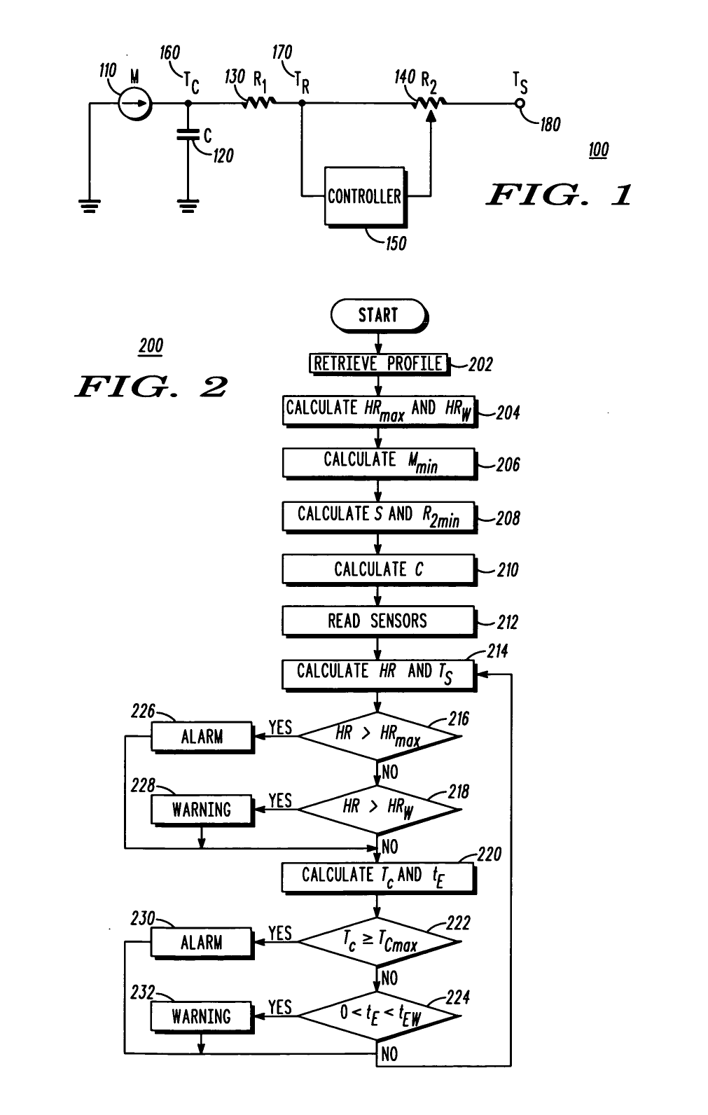

Referring now to the drawings, and in particular FIG. 1, a heat balance model used in determining parameters for monitoring heat stress in accordance with embodiments of the present invention is shown and indicated generally at 100. The heat balance model is described by way of a human body. However, the concepts related thereto are equally applicable to other living organisms, such as livestock. Thus, it should be understood that the teachings herein are not limited to applications involving a human but can be easily expanded to other applications.

Before describing the heat balance model 100, it would be beneficial to the understanding of embodiments of the invention to first describe the heat dissipation mechanism of a human body. The human body continuously produces heat that it uses to maintain its constant temperature. The amount of heat produced increases significantly when someone performs a physical activity, and the body removes excess heat through radiation, convection and sweat evaporation. At rest, the human body requires approximately 0.3 liters (l) of oxygen and generates (on average) 90 kcal/h. The heart rate required to meet this oxygen demand ranges from 60 to 80 beats per minute for most of the mature human population. At maximum effort the oxygen demand is in the range 2.5-3.5 l/min, depending on age, gender and degree of fitness.

Blood flow rate is proportional to the heart rate. Therefore, the body's heat production has a linear dependency on the heart rate. As a response of subcutaneous vasodilatation produced by increased skin temperature, the heart rate increases to maintain the blood flow in muscles and internal organs. This is in addition to the heart rate response to the oxygen demand for performing physical activities.

The temperature regulating center of the body is located in the brain region called the hypothalamus. It responds to increases in its own temperature and to signals from thermal receptors in the skin by increasing the blood flow to the skin and sweating. The temperature set point for human heat regulation is approximately 37° C., but can vary to a certain extent from person to person. It can also change with the time of the day, decreasing about 1° C. at night from the daytime peak value. The skin temperature set point is around 34° C.

It has been observed that body core temperature, T_{C}, is not exactly equal to the set point for human heat regulation, T_{R}, but increases slightly with the effort, which was explained by a certain resistance to the heat flow from the heat source to the internal thermal receptors, which causes a temperature drop resulting in the difference. The resistance to the heat flow to the skin is variable as the blood flow is adjusted by the thermoregulation mechanisms of the body, but has a lower limit, beyond which the difference between T_{R} and the skin temperature, T_{S}, cannot decrease significantly.

The heat dissipation mechanism can be more easily understood by an electrical analogy, for instance, through the use of the heat balance model 100 illustrated in FIG. 1. In this model, a current source 110 represents metabolic heat production rate, M, a capacitor 120 represents body heat capacity, C, a point T_{C} (160) represents body core temperature, a point T_{R} (170) represents temperature of or as measured by body heat sensors, a point T_{S} (180) represents skin surface temperature, and resistors. 130 (R₁) and 140 (R₂) represent heat flow resistances. A regulating block or controller 150 represents the regulating function of the hypothalamus. An exemplary calculation process or algorithm to determine when to alert a user that he or she is at risk of developing heat related disorders if remaining in a current environment is explained below based on this model, wherein:
c represents body specific heat;
C represents body heat capacity;
H represents body height;
HR represents heart rate;
HRₘₐₓ represents maximum heart rate;
HRₘᵢₙ represents resting heart rate;
HR_{W} represents warning threshold for heart rate;
ΔHRₜₑₘₚ represents heart rate increase due to temperature;
M represents metabolic heat generated by body;
Mₘᵢₙ represents metabolic heat generated by body at rest;
Q represents heat removal rate;
R₁ represents heat resistance to flow between core and heat sensors;
R₂ represents heat resistance to flow between heat sensors and skin;
R₂ₘᵢₙ represents minimum of R₂;
S represents skin surface area;
T_{C} represents body core temperature;
T_{Cmax} represents maximum safe core temperature;
t_{E} represents endurance time (predicted time for reaching T_{Cmax});
t_{EW} represents endurance time warning threshold;
T_{R} represents temperature of body heat sensors;
T_{S} represents skin surface temperature;
T₀ represents body heat regulation set temperature; and
W represents body weight.

At normal temperatures, e.g., less than 40° C. body temperature, there is a linear relationship between heart rate and metabolic heat generated by the body. Also, heart rate increases when skin surface temperature increases. Published data indicate that the heart rate increase is well described by an exponential function of the skin surface temperature:
ΔHRₜₑₘₚ=ae^{bT}^{_{S}} (1)
where a and b are constants. It should be obvious to those skilled in the art, however, that other functions relating heart rate increase to skin surface temperature and that fit well with experimental data can alternatively be used.

Knowing T_{S} we can calculate ΔHRₜₑₘₚ.

If HR is measured and the resting heart rate, HRₘᵢₙ, is known, we can calculate the metabolic heat generation rate:
M=m×(HR−HRₘᵢₙΔHRₜₑₘₚ)+Mₘᵢₙ (2)
where the metabolic heat generation rate at rest, Mₘᵢₙ, and m, are constants.

Assuming no heat is generated in the heat flow path from the body core to the skin, the heat removal rate continuity requires that

$$Q = \frac{{T}_{C} - {T}_{R}}{{R}_{1}} = \frac{{T}_{R} - {T}_{S}}{{R}_{2}} . \qquad ( 3 )$$

R₁ is a constant, and R₂ can be calculated from the above equation. However, the value of R₂ is lower bound by a minimum value, wherein:

$${R}_{2} = Max ( {R}_{1} \frac{{T}_{0} - {T}_{S}}{{T}_{C} - {T}_{0}} , {R}_{2 min} ) . \qquad ( 4 )$$
R₂ₘᵢₙ is inversely proportional to the skin surface area, S, therefore we can write

$${R}_{2 min} = \frac{k}{S} \qquad ( 5 )$$
where k is a constant.

If T_{S} and T_{C} are known R₂ can be calculated from the equations (4) and (5) and we can then calculate the heat removal rate

$$Q = \frac{{T}_{C} - {T}_{S}}{{R}_{1} + {R}_{2}} . \qquad ( 6 )$$

The body heat capacity is the body weight multiplied by the average tissue specific heat:
C=cW (7)

Once this is known we can calculate the core temperature increase during the time interval Δt, based on body heat balance:

$$Δ {T}_{C} = Δ t \frac{M - Q}{C} \qquad ( 8 )$$

The initial core temperature is generally not known, but can be assumed to be 37° C. at rest.

If the core temperature at time t is known, the temperature at time t+Δt
T_{C}(t+Δt)=T_{C}(t)+ΔT_{C} (9)
which is the current core temperature.

We can also calculate the predicted time until a safe limit of the core temperature is reached

$${t}_{E} = Δ t \frac{ {T}_{C max} - {T}_{C} ( t + Δ t )}{Δ {T}_{C}} . \qquad ( 10 )$$

Increase of body core temperature to 39° C. should normally be permitted only briefly. Therefore, we can take this value for T_{Cmax}. It is further desirable that the user is warned when t_{E} falls below a limit t_{EW} set for the specific activity, to give him time to take action. Moreover, an alarm can be generated when core temperature reaches T_{Cmax}, informing him that he is in immediate danger.

The constants used in the equations above have limited ranges, and default values (based on population averages) can be used, but the accuracy of the calculations can be increased if their values are measured for each user based on a personal profile created for the user. At a minimum, the heart rate when the person is at rest, HRₘᵢₙ, is recorded and entered into the personal profile. The person's weight is also important since the metabolic heat generation rate at rest can be calculated from:
Mₘᵢₙ[kcal/h]=1.05×W[kg] (11)
Height can be used to calculate the skin surface area from the equation:
S[m²]=W[kg]^{0.425}×H[cm]^{0.725} (12)

To determine the constant m in equation (2) one can measure the heart rate during a standardized (or predetermined) exercise, where M is known, and at a skin temperature less than 33° C. For example, walking on a horizontal treadmill at a speed V, the metabolic heat generation rate is:
M[kcal/h]=0.3×W[kg]×V[m/min] (13)

By using the value of Mₘᵢₙ calculated from equation (11) and HRₘᵢₙ one can calculate m from equation (2), wherein:

$$m = \frac{M - {M}_{min}}{HR - {HR}_{min}} . \qquad ( 14 )$$

This will account for the person's degree of fitness.

To determine the constants a and b in equation (1) one can measure the heart rate during a standardized exercise, and at a few skin temperatures above 35° C. From equation (2):

$$Δ {HR}_{temp} = HR - {HR}_{min} - \frac{M - {M}_{min}}{m} \qquad ( 15 )$$

and from (1)
ln(ΔHRₜₑₘₚ)=bT_{S}+a (16)
wherein a and b can be calculated by regression. This will account for the person's degree of acclimatization. Finally, age and gender may be used to calculate the maximum heart rate, HRₘₐₓ. For males:
HRₘₐₓ[beats/min.]=205−A[years]/2, and (17)
for females:
HRₘₐₓ[beats/min.]=220−A[years] (18)

The maximum heart rate does not enter in the equations above, but it is an absolute limit for their validity. When the heart rate approaches this limit the heart is overstressed, putting the person at risk. Also, the oxygen supply is not sufficient to sustain aerobic work. Therefore the user should be warned when a certain limit HR_{W} is reached, for example 75% of HRₘₐₓ.

The calculations presented above can be performed, for example, by a processing device executing software stored in a memory. Such an algorithm is summarized by the flow diagram illustrated by reference to FIG. 2. In general, the method performed in accordance with above calculations comprises the steps of: determining a plurality of physiological parameters including measuring at least heart rate and skin surface temperature of a user; calculating at least body core temperature of the user as a function of the plurality of physiological parameters using a predetermined algorithm; comparing at least heart rate and body core temperature to corresponding maximum thresholds; and alerting the user when at least one of heart rate and body core temperature exceeds the corresponding maximum threshold. As stated above, optionally a user's personal profile can be taken into consideration in the calculations to generate more accurate calculations. Method 200 represents one exemplary method that includes a user's profile in the algorithm.

When the program is started, it retrieves in a step 202 a user's anthropometric data (also referred to herein as a user's personal profile) from memory, or in addition to and/or alternatively may prompt the user to enter a portion or all of her personal profile. For example, the gender, weight, height, heart rate at rest, the constants a, b, and m are retrieved. If any of the constants are not known, a default value is stored in memory and used. In a step 204 HRₘₐₓ is calculated according to equations (17) or (18), as well as HR_{W}. In a step 206 Mm is calculated according to equation (11). In a step 208 S and R₂ₘᵢₙ are calculated according to equations (12) and (5). In a step 210 C is calculated according to equation (7), where water specific heat can be taken as a value for c. At a step 212, sensors associated with the user's current heart rate (e.g., sensors that measure the user's pulse rate) and associated with the user's skin surface temperature are read from, for instance, pulse and temperature sensors. In a step 214 sensor readings are converted to skin surface temperature and heart rate, and other calculations may be performed, specific to the physical implementation, such as averaging among each type of sensors, averaging over time, etc.

At a step 216 the heart rate is compared with the maximum heart rate, HRₘₐₓ, and an alarm is sent, 226, to the user if the heart rate exceeds this threshold. At a step 218 the heart rate is compared with a threshold HR_{W} that is typically less than HRₘₐₓ (for example 90% of the maximum threshold) and a warning is sent, 228, to the user if the heart rate exceeds this warning threshold. Using a warning threshold alerts the user that he is approaching the maximum threshold, thereby, giving the user additional time to prepare to leave the environment if the alarm is triggered when the maximum threshold is exceeded. Similarly, in a step 220 body core temperature and endurance time are updated according to equations (9) and (10). At a step 222 the core temperature, T_{C}, is compared to a maximum core temperature, T_{Cmax}, and an alarm is sent, 230, to the user if the body core temperature exceeds this threshold. At a step 224 the endurance time, t_{E}, is compared to a threshold t_{EW} and a warning is sent, 232, to the user if the endurance time exceeds this threshold. Using an endurance time threshold gives the user an indication of the amount of time remaining before the maximum limit for body core temperature is exceeded. It should be noted that in this embodiment only positive values of t_{E} are considered, since t_{E} can become negative when the core temperature decreases, which does not justify a warning.

The above-described process can be implemented with any suitable system that includes: a plurality of sensors measuring a plurality of physiological parameters including at least heart rate and skin surface temperature of a user; typically at least one of a memory and a user input terminal for storing and/or entering a user profile and for storing an algorithm for executing by a processing device; a processing device performing the steps of calculating at least body core temperature of the user as a function of the plurality of physiological parameters using a predetermined algorithm, comparing at least heart rate and body core temperature to corresponding maximum thresholds, and determining when at least one of heart rate and body core temperature exceeds the corresponding maximum threshold; and alarm apparatus alerting the user when at least one of heart rate and body core temperature exceeds the corresponding maximum threshold.

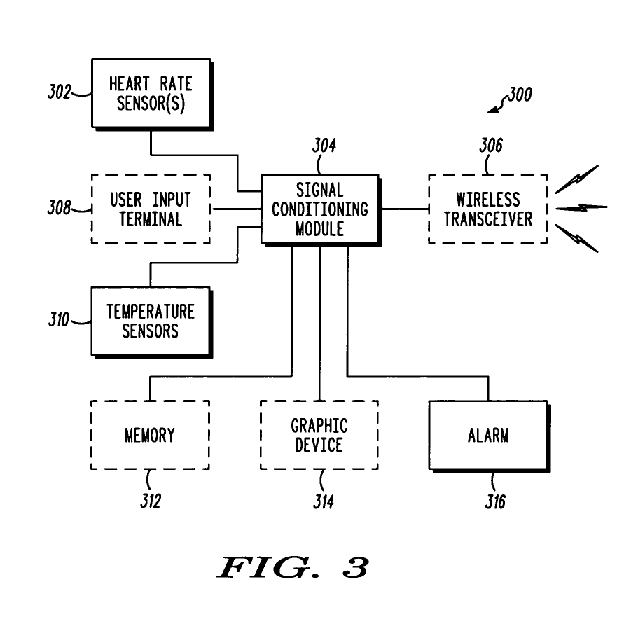

FIG. 3 illustrates an exemplary physiological monitoring system 300 for implementing the embodiments described herein. The system 300 includes at least one wearable heart rate sensor 302, such as an acoustic or optical sensor for instance, a signal-conditioning module 304 (also generally referred to herein as a processing device, as any conventional processing device may be used for its implementation), temperature sensors 310 and alarm apparatus 316. Heart rate signals from a body (such as a user's pulse rate) are detected by the sensors 302, and the sensors 310 detect temperature associated with skin surface temperature. The signals from these sensors are received in and processed by the signal conditioner 304, which also performs the calculations as described above by reference to FIGS. 1 and 2. Alarm apparatus alerts the user when any of the monitored physiological parameters exceeds a threshold. The alarm apparatus can, for example, provide an auditory alert and/or signals for a visual alert to the user. Different sounds or visual indicators (e.g., colored lights) can be used to distinguish between a warning and an indication that a maximum threshold has been exceeded.

System 300 may also optionally comprise a wireless transceiver 306 for sending to a monitoring source such as a dispatch personnel, a server collecting data, etc., for example, one or more of the measured or calculated physiological parameters, and/or indications of when any of the monitored parameters exceeds a threshold. System 300 may further optionally comprise: a graphical device 314 for prompting the user to input certain portions of her user profile and possibly displaying alerts visible to the user and corresponding to alarms and/or warning being triggered by monitored parameters exceeding a warning or maximum threshold; memory apparatus 312 for storing, for example, at least portions of the user's profile, the above-described algorithm used in monitoring the physiological parameters, all of the thresholds used and any default parameter used in the predetermined algorithm; and a user input terminal 308 to enable the user to respond to prompts for data and information.

The system 300 thus enables remote, real-time auscultation of various vital parameters of personnel that are experiencing high-stress. A remote command center, for example, can monitor firefighters in a burning building and determine instantly when a particular firefighter should exit the building or needs assistance, or when a shift of firefighters should be rotated away from a hazardous environment because of extreme stress. Similarly, the real-time health of personnel in other hazardous occupations such as law enforcement, mining, diving, and the military can be monitored remotely. The effects of high stress and extreme heat, such as heat exhaustion and heat stroke, can therefore be avoided or remedied through remote auscultation. Such remote auscultation may further benefit others such as athletes in training and people with fragile health including the very young and the elderly.

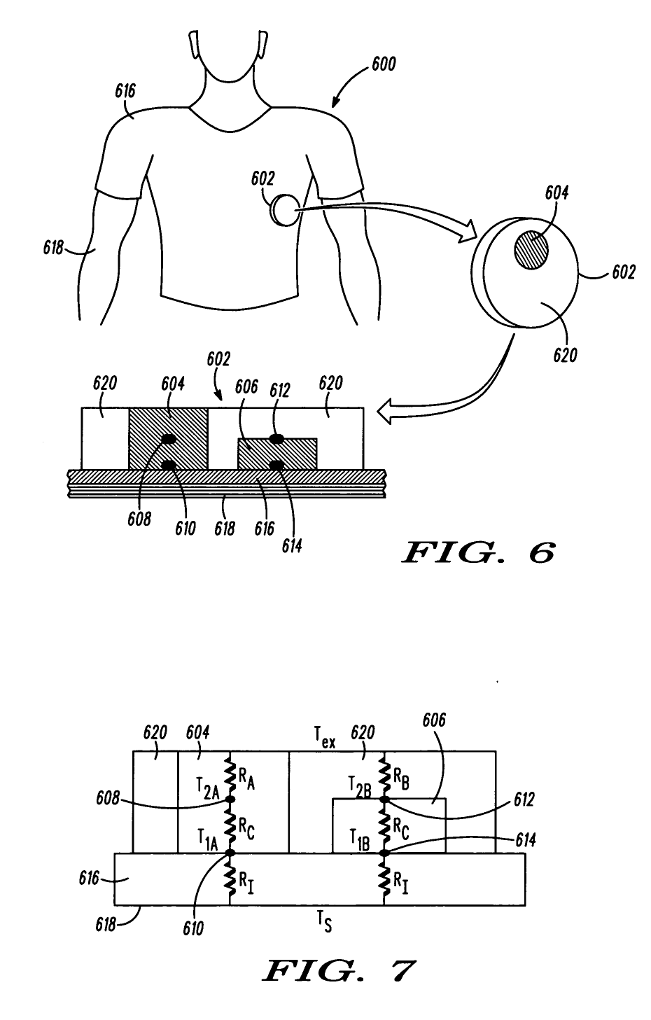

The system can measure the heart rate and skin temperature by means of sensors in direct contact with the body, i.e., sensors that touch the skin, are inserted into or beneath the skin or are inserted into a body cavity such as an ear. However, the system can be easier to utilize if it does not require direct contact to the user's skin. This can be achieved by replacing heart rate sensors based on skin contact electrodes with a suitable acoustic sensor, or multiplicity of sensors. Capacitive coupled electrodes can also be used. Moreover, temperature sensors that do not require direct contact with a user's skin, such as the sensors described below by reference to FIGS. 6 and 7, may be used.

Where direct skin contact is not required, various alternative designs of the present physiological monitoring system 300 are made possible, which are comfortable to wear and appropriate for various occupational conditions. For example, the system 300 can be incorporated into jackets and vests that are generally worn over undergarments such as T-shirts. The present physiological monitoring system may likewise be attached to an article of clothing such as, for instance, bulletproof vests, fire retardant jackets, diving suits, law enforcement uniforms, military uniforms, mining uniforms, athletic uniforms, trousers, patient gowns, clothing for children and the elderly, etc. A harness design 400 described below is an example of one such design, and other designs are described in more detail below.

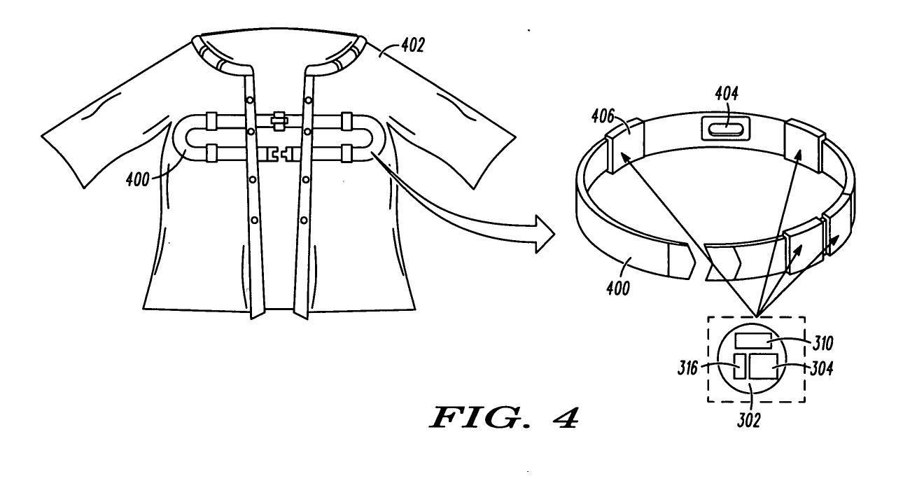

Referring to FIG. 4, there is a schematic diagram illustrating a wearable harness 400 that incorporates the system 300 according to a specific embodiment of the present invention. The harness 400 is designed to be removably attached to clothing such as a firefighter's jacket 402. A loop 404 at the back of the harness 400 enables secure attachment of the harness 400 to the jacket 402. The harness 400 includes multiple heart rate sensors 302, and additional devices such as temperature sensors 310, a signal conditioning module 304, and alarm apparatus 316. When worn by a person, some of the sensors 302 can be located near the person's back, for example to measure respiratory sounds, and other sensors are located near the person's chest, for example to measure cardiologic sounds. Other sensors, such as a respiration sensor 406 attached to a back portion of the harness 400, may also be incorporated into the harness 400.

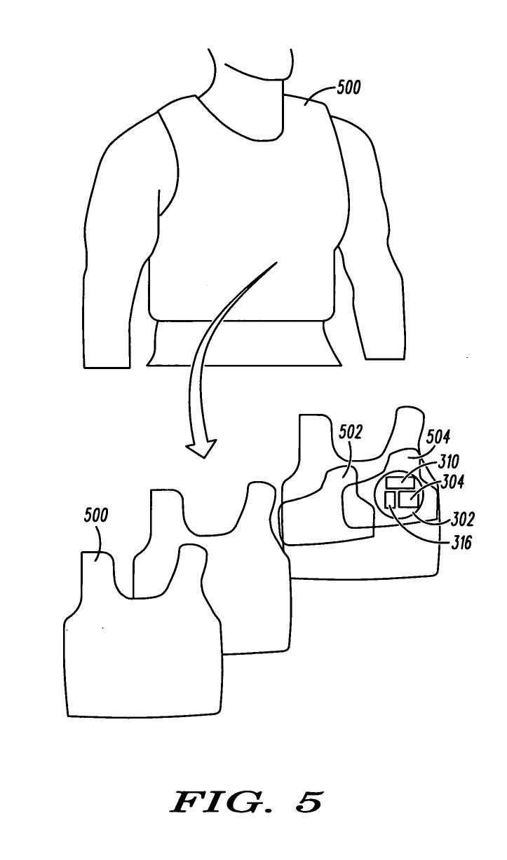

Referring to FIG. 5, there is a schematic diagram illustrating a further embodiment of the present invention incorporated into a vest 500. Here, sensors 302 and 310, a signal conditioning module 304 and alarm apparatus 316, among optionally other instrumentation, are incorporated into the vest 500 on top of a relatively large-area acoustic impedance matching element 504. A foam pad 502 is then, ideally, placed on top of the instrumentation to protect it from external forces and impacts and to insulate it from outside noise—thereby increasing the sensitivity of the sensors 302. The physiological monitoring system 300 of the present invention therefore may be incorporated into existing occupational clothing, such as bulletproof vests worn by police officers. The armor protection of a bulletproof vest may be included in the multiple plys of the vest 500.

Measuring skin temperature through a thin layer of insulation, such as a shirt, can be achieved by means of a probe that compensates for temperature drop through the insulation. In general the temperature probe comprises: a first portion comprising a thermally conductive material having a first plurality of temperature measuring devices coupled thereto and having a first face adjacent to a first surface of the sensor; a second portion comprising a thermally conductive material having a second plurality of temperature measuring devices coupled thereto and having a second face adjacent to the first surface of the sensor; and a third portion coupled between the first and second portions and substantially thermally isolating the first portion from the second portion, wherein upon the first surface of the sensor contacting a second surface that is adjacent to a user's skin surface a difference in heat flux between the first and second portions is generated and measurable based on the first and second plurality of temperature measuring devices, the measurable difference in heat flux for use in determining skin surface temperature of the user.

FIG. 6 shows an example of an embodiment of a temperature probe 602 used to monitor skin surface temperature of a person 600. The temperature probe 602 is placed with a lower face in contact with an accessible surface adjacent to the body whose temperature is to be measured. For example, the probe can be placed on top of a thin clothing layer 616, such as a shirt, which is adjacent to the user's skin 618. The probe includes two sections 604 and 606. The first section 604 comprises a thermally conductive material such as, for instance, a plastic material that has a higher conductivity than the surrounding insulating material, having two thermistors 608 and 610 embedded therein, one close to the lower surface and another one between (in one embodiment midway between) an upper and a lower face of the section 604, with thermistor 608 being substantially directly above thermistor 610 within tolerances based on the mode of manufacturing the temperature probes. The second section 606 comprises the same conductive material also having two thermistors embedded therein at substantially the same distance as the thermistors in the first section, with thermistor 608 being substantially directly above thermistor 610 within tolerances based on the mode of manufacturing the temperature probes. Those skilled in the art will realize that in alternative embodiments such distances between the thermistors of each section may be difference, realizing that such difference can easily be factored in the calculations below to determine skin surface temperature.

Sections 604 and 606 are separated by a substantially thermally isolating material 620 such as, for instance, a polystyrene or a polyurethane foam. Moreover, both sections have lateral surfaces surrounded by thermal insulation 620, with section 606 having its face that is not adjacent to the insulating material 616 also surrounded by thermally isolating material 620. In the above-described exemplary temperature probe, the temperature measuring devices used were thermistors. However, skilled artisans will realize other temperature measuring devices can be used such as, for instance, thermocouples, semiconductor temperature sensors (e.g., diode sensors) and resistance temperature detectors (RTDs) without departing from the scope of the teachings herein.

FIG. 7 illustrates the operating principle of the temperature probe 602. It is assumed that the probe is not loading the source, meaning that the heat flux is low enough in both sections and does not modify the source temperature, T_{S}. Elements comprising the circuit model of sensor 602 correspond to the elements of probe 602 described by reference to FIG. 6 and comprise:
R_{A} representing heat flow resistance to external surface in section A;
R_{B} representing heat flow resistance to external surface in section B;
R_{C} representing heat flow resistance from contact surface to interior;
R₁ representing heat flow resistance in clothing material;
T_{S} representing skin surface temperature;
Tₑ representing ambient temperature;
T_{1A} representing section A surface temperature;
T_{2A} representing section A internal temperature;
T_{1B} representing section B surface temperature; and
T_{2B} representing section B internal temperature.

At a certain external temperature, Tₑₓ, the temperatures of the contact surfaces are T_{2A} and T_{2B}, for the conductive and insulated sections of the probe, respectively. The inner temperatures of each section are T_{1A} and T_{1B}.

Temperature T_{1A} is approximately midway between Tₑₓ and T_{2A}, while T_{1B} will be closer to T_{2B}, due to the insulating layer of section B. The lateral insulation enables the heat flux to be properly oriented along the vertical axis in each section. At steady state, the heat flux is constant along the vertical axis, therefore

$$\begin{matrix}\frac{( {T}_{S} - {T}_{1 A} )}{{R}_{1}} = \frac{( {T}_{1 A} - {T}_{2 A} )}{{R}_{C}} and & ( 19 a ) \\ \frac{( {T}_{S} - {T}_{1 B} )}{{R}_{1}} = \frac{( {T}_{1 B} - {T}_{2 B} )}{{R}_{C}} , & ( 19 b )\end{matrix}$$

where R₁ is the heat resistance of the clothing layer and R_{C} is the resistance of the conductive material in the probe between the two thermistors of each section. R_{C} is usually unknown but by taking the ratio of equations (19), designated by K, R_{C} is eliminated:

$$K = \frac{( {T}_{S} - {T}_{1 A} )}{( {T}_{S} - {T}_{1 B} )} = \frac{( {T}_{1 A} - {T}_{2 A} )}{( {T}_{1 B} - {T}_{2 B} )} . \qquad ( 20 )$$

Since T_{1A}, T_{2A}, T_{1B}, and T_{2B} are measured, K can be calculated. Then we can calculate T_{S} as

$${T}_{S} = \frac{{T}_{1 A} - {KT}_{1 B}}{1 - K} . \qquad ( 21 )$$

Thus, we can measure the temperature of the heat source even without a perfect thermal contact which happens when a thin layer of clothing is present. It should be noted that when K equals one, equation (21) cannot be used, but this happens only if the flux becomes zero, therefore all temperatures are equal to T_{S}. The resistance R_{C} was assumed the same in both sections, but minor variations can be eliminated by probe calibration.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

What is claimed is:

1. A method for monitoring heat stress of a user comprising the steps of:
determining a plurality of physiological parameters including measuring at least heart rate and skin surface temperature of a user;
calculating at least body core temperature of the user as a function of the plurality of physiological parameters using a predetermined algorithm;
comparing at least heart rate and body core temperature to corresponding maximum thresholds; and
alerting the user when at least one of heart rate and body core temperature exceeds the corresponding maximum threshold.

2. The method of claim 1 further comprising the step of retrieving a profile of the user comprising personal characteristics associated with the user, wherein body core temperature is calculated further based on the user's profile.

3. The method of claim 2, wherein the user's profile comprises at least one of a minimum heart rate for the user at rest, the user's weight, the user's height, the user's age, the user's gender and a measure of acclimatization for the user.

4. The method of claim 3, wherein the measure of acclimatization for the user is calculated based on a measure of at least one of the user's heart rate and skin surface temperature while the user is performing a predetermined exercise.

5. The method of claim 1 further comprising the step of alerting an external monitoring source other than the user of at least one of the plurality of measured physiological parameters and the calculated body core temperature for the user.

6. The method of claim 1, wherein alerting the user comprises triggering at least one of an alarm visible to the user and an alarm audible to the user.

7. The method of claim 1 further comprising the steps of:
comparing at least heart rate and body core temperature to corresponding warning thresholds that are less than the maximum thresholds; and
alerting the user when at least one of heart rate and body core temperature exceeds the corresponding warning threshold.

8. The method of claim 7 further comprising the step of alerting an external monitoring source other than the user when at least one of heart rate and body core temperature exceeds the corresponding warning threshold.

9. The method of claim 1 further comprising the step of alerting an external monitoring source other than the user when at least one of heart rate and body core temperature exceeds the corresponding maximum threshold.

10. A system for monitoring heat stress of a user comprising:
a plurality of sensors measuring a plurality of physiological parameters including at least heart rate and skin surface temperature of a user;
a processing device performing the steps of:
calculating at least body core temperature of the user as a function of the plurality of physiological parameters using a predetermined algorithm;
comparing at least heart rate and body core temperature to corresponding maximum thresholds; and
determining when at least one of heart rate and body core temperature exceeds the corresponding maximum threshold; and
alarm apparatus alerting the user when at least one of heart rate and body core temperature exceeds the corresponding maximum threshold.

11. The system of claim 10, wherein at least one of the plurality of sensors comprises a sensor that does not require direct physical contact to the user's skin in order to measure its corresponding physiological parameter.

12. The system of claim 11, wherein at least one of the sensors not requiring direct physical contact to the user's skin is a skin surface temperature probe comprising:
a first portion comprising a thermally conductive material having a first and second thermistor coupled thereto and having a first face adjacent to a first surface of the sensor, wherein the first thermistor is located at a first distance from the first surface of the sensor and the second thermistor is located above the first thermistor at a second distance from the first surface of the sensor that is greater than the first distance;
a second portion comprising a thermally conductive material having a third and fourth thermistor coupled thereto and having a second face adjacent to the first surface of the sensor, wherein the third thermistor is located at a third distance from the first surface of the sensor that is substantially equal to the first distance and the fourth thermistor is located above the third thermistor at a fourth distance from the first surface of the sensor that is substantially equal to the second distance; and
a third portion coupled between the first and second portions and substantially thermally isolating the first portion from the second portion, wherein upon the first surface of the sensor contacting a second surface that is adjacent to a user's skin surface a difference in heat flux between the first and second portions is generated and measurable based on positioning of the first, second, third and fourth thermistors, the measurable difference in heat flux for use in determining skin surface temperature of the user.

13. The system of claim 10 further comprising a wireless transmitter forwarding to a monitoring source other than the user at least one of the plurality of measured physiological parameters, the calculated body core temperature and the results of determining when at least one of heart rate and body core temperature exceeds the corresponding maximum threshold.

14. The system of claim 10 further comprising memory apparatus storing at least one of the predetermined algorithm, the maximum thresholds and default parameters used in the predetermined algorithm.

15. The system of claim 14, wherein the memory apparatus further stores at least a portion of a profile of the user comprising personal characteristics associated with the user and used by the processor to calculate the user's body core temperature.

16. A sensor for measuring skin surface temperature of a user comprising:
a first portion comprising a thermally conductive material having a first plurality of temperature measuring devices coupled thereto and having a first face adjacent to a first surface of the sensor;
a second portion comprising a thermally conductive material having a second plurality of temperature measuring devices coupled thereto and having a second face adjacent to the first surface of the sensor; and
a third portion coupled between the first and second portions and substantially thermally isolating the first portion from the second portion, wherein upon the first surface of the sensor contacting a second surface that is adjacent to a user's skin surface a difference in heat flux between the first and second portions is generated and measurable based on the first and second plurality of temperature measuring devices, the measurable difference in heat flux for use in determining skin surface temperature of the user.

17. The sensor of claim 16, wherein the first plurality of temperature measuring devices comprises a first plurality of thermistors, and the second plurality of temperature measuring devices comprises a second plurality of thermistors.

18. The sensor of claim 17, wherein the first plurality of thermistors includes a first and second thermistor having a distance therebetween and the second plurality of thermistors includes a third and fourth thermistor having a distance therebetween that is substantially the same as the distance between the first and second thermistors.

19. The sensor of claim 18, wherein:
the first thermistor is located at a first distance from the first surface of the sensor;
the second thermistor is located above the first thermistor at a second distance from the first surface of the sensor that is greater than the first distance;
the third thermistor is located at a third distance from the first surface of the sensor that is substantially equal to the first distance; and
the fourth thermistor is located above the third thermistor at a fourth distance from the first surface of the sensor that is substantially equal to the second distance.

20. The sensor of claim 19, wherein the second thermistor is located substantially directly above the first thermistor, and the fourth thermistor is located substantially directly above the third thermistor.
